(19) United States
(12) Patent Application Publication
(10) Pub. No.: US 2010/0291174 A1
(43) Pub. Date: Nov. 18, 2010

(54) BIOLOGICALLY ACTIVE DEVICE AND METHOD FOR ITS PRODUCTION

(76) Inventors: Stephan Barcikowski, Hannover (DE); Thomas Lenarz, Hannover (DE); Timo Stoever, Hannover (DE)

Correspondence Address: FLYNN THIEL BOUTELL & TANIS, P.C., 2026 RAMBLING ROAD, KALAMAZOO, MI, 49008-1631, US

(21) Appl. No.: 12/449,311

(22) PCT Filed: Feb. 1, 2008
(86) PCT No.: PCT/EP2008/000803

§ 371 Date: Apr. 15, 2010

(30) Foreign Application Priority Data
Feb. 6, 2007 (DE) 10 2007 005 817.0

(51) Int. Cl. A61F 2/00; A61K 33/42; A61K 33/38; A61K 33/34; A61K 33/30; A61K 9/00; A61F 2/82; A61F 2/24; A61F 2/06; A61M 25/00; A61F 2/18; A61P 41/00; B29C 35/08; C12N 5/07

(52) U.S. Cl. 424/423; 424/602; 424/618; 424/638; 424/641; 424/422; 264/400; 435/377; 977/773

(57) ABSTRACT

The present invention is directed to a biologically active device with a main body made from a polymer, in which bioactive nanoparticles of one or several materials are embedded, wherein the nanoparticles of at least one material proliferatively act on a biological material contacted by the device, and wherein nanoparticles of a different material act in an anti-proliferative manner on biological material in the ambience of the device. The invention is also directed to a method of manufacturing a biologically active device with a main body made from a polymer, wherein nanoparticles of several different materials are dispersed in an injection-moldable fluid, and the fluid is shaped into the polymer main body by means of injection molding and curing, such that the nanoparticles are dispersed in the bulk of the polymer main body. According to the invention, the nanoparticles are generated by arranging at least two substrates of different material in a vessel filled with a fluid material, and by generating the nanoparticles by abrasion from the surface of the substrates in the fluid with laser radiation.

### I. FIELD OF THE INVENTION

The present invention concerns a biologically active device, as well as a method for manufacturing same. In the context of this document, a “biologically active device” is a device which is configured to affect a surrounding biological material, or to interact with such biological material. A special application of such a biologically active device are medical devices, apparatus and instruments.

### II. BACKGROUND OF THE INVENTION

A particular example of a biologically active device is an implant which is implanted into a human or animal body. When inserting an implant into the body of a patient, there is usually the danger that bacteria assemble on the implant, which can trigger an immune reaction and an inflammation of the tissue in which the implant is embedded. Another problem may consist in the implantation leading to an increased growth of connective tissue. The new connective tissue cells overlie the implant and impede the delivery of electrical or optical signals from the implant (e.g. from pacemakers, cochlear- or neuro-implants) to nerve cells in the environment of the implant.

As an anti-infection protection, it is known to provide a solid main body of an implant with a surface coating in or on which anti-microbial, anti-bacterial, or anti-proliferative (i.e. cell growth hampering) substances are provided.

For example, DE 102 43 132 A1 describes an anti-infectious titanium oxide coating for an implant, which may release anti-bacterial metal ions.

DE 197 56 790 A1 suggests to embed anti-microbial silver particles with a grain size below 20 nm into a polymer.

According to DE 103 53 756 A1, a solid main body of an implant is provided with a double coating. An inner reservoir layer comprises biocidal (i.e. cell damaging) agents with a grain size below 50 nm. The biocidal agent may comprise silver, copper, or zinc. An outer “transport control layer” serves to control or reduce the delivery of this agent.

EP 1 131 114 B1 suggests to coat the surface of an implant with a polymer layer, in which a tissue reaction modificator is embedded.

US 2004/0215338 A1 is directed to a coated stent graft. A coating of the stent graft body comprises bio-active nanoparticles, which exclusively deliver anti-proliferative substances.

WO 2006/096791 A1 describes an implant which serves as a framework for the tissue regeneration. The main body of the implant is made from polymer nanofibers, on or between which nanoparticles are arranged which deliver bioactive molecules.

WO 2006/068838 A2 discloses medical implants with a nanoporous or “nano-textured” surface, into which no nanoparticles, but rather cell adhesion supporting biomolecules are embedded.

WO 2003/049795 A2 describes several possibilities for the manufacturing of implants, in which a “nanoparticulate filler” is embedded into a matrix.

US 2006/0177379 A1 discloses a material for implants which comprises both a “therapeutically active agent” (for example in the form of nanoparticles) as well as a “signal generating agent”, which agents are delivered into the environment together. By means of physical or chemical measurements of the “signal generating agent” (for example, via x-rays or spectroscopy), the delivery of the therapeuticum can be monitored.

US 2005/0095267 A1 suggests a polymer coating comprising nanoparticles for implants. However, this document relates exclusively to nanoparticles which release anti-proliferatively active substances.

Finally, US 2006/0188543 A1 is directed to cardiovascular stents with a lipid monolayer coating which comprises nanoparticles from a biodegradable and/or bioresorbable polymer, these nanoparticles in turn carrying an agent. Since the aim of this implant is the prevention of vascular restenosis, this document exclusively refers to anti-proliferative agents.

It is the object of the present invention to provide a biologically active device which is easy to manufacture, can be adapted without significant effort to different surroundings or requirements, and may interact ideally with surrounding biological tissue.

### SUMMARY OF THE INVENTION

This object is solved by a biologically active device with the features of claim 1, or by a method for manufacturing a biologically active device with the features of claim 19.

The biologically active device of the present invention comprises “bioactive” nanoparticles, i.e. nanoparticles which release substances which can interact with biological receptors in their proximity or ambience. In this context, the term “nanoparticles” refers to particles having dimensions in the sub-micrometer range. Due to their small size, such nanoparticles have a relatively large surface, across which they can deliver agents (in the case of metal nanoparticles: e.g. ions). At the same time, however, the volume of the nanoparticles turns them into a considerable reservoir for the substances to be released, in particular in comparison to simple molecules.

In contradiction to almost all conventional implants, the bioactive nanoparticles are arranged, in the biologically active device of the present invention, not exclusively in a coating of a main body, but embedded in the main body itself, wherein the main body is made from a polymer. This allows a rather simple, compact structure of the biologically active device, since the main body of the device defines not only the shape of the device, but also its bioactive function.

In particular, the biologically active device of the present invention is different from conventional implants or other biologically active devices by comprising nanoparticles from different substances which provide for different, on first sight even contradictory-appearing effects on a biological material by which the device may be contacted. While the nanoparticles of one material release a substance having a proliferative effect, other nanoparticles release a substance which has an anti-proliferative or anti-adherent effect on biological material in the ambience of the device. In the context of the invention, “proliferative” or “proliferation” does not only mean any beneficial effect on cell or tissue growth, but any positive effect on the biological material surrounding the device, e.g. also an encouragement of cell adhesion, i.e. the attachment of cells on the device. Correspondingly, “anti-proliferative” means any negative effects on biological material, including antibacterial or antimicrobial effects. An “anti-adherent” effect means that the attachment of biological material, including biofilms, to the device is delayed, retarded, hindered or even completely prevented.

Regarding an application of the inventive device as an implant, this contradicts the conventional opinion that an implant must be provided exclusively with anti-proliferative (e.g. cell growth reducing) substances, since otherwise an undesired, inflammation-provoking multiplication of germs would be supported, or exclusively with proliferative (e.g. cell growth supporting) substances, since otherwise the two mutually contradicting effects would annul themselves. Surprisingly, however, it could be shown that the acceptance of a biologically active device, for example as an implant, could be significantly increased if the device releases both “proliferative” and “anti-proliferative” substances or combinations of substances. A particularly beneficial effect achievable in this way is the selectivity for certain cell types by supporting the desired type or tissue (e.g. endothelial cells, fibroblasts, or nerve cells) while simultaneously suppressing the undesired type (tissue excrescence, bacteria). In this way, for example, tissue excrescence may selectively be suppressed, and simultaneously a neurotrophic effect (e.g. from a neuro- or cochlear-implant) may be achieved; or the settlement and multiplication of inflammation germs may be suppressed, while simultaneously supporting the implant integration in the surrounding tissue. This increased acceptance or long-term stability is remarkable, in particular with implants which are still in contact with air after being applied to the patient—cochlear implants, for example, as well as cannulae or (port-)catheters, which conventionally show a strong tendency to inflammation.

Tests have shown that the differing effects of such a combination of proliferative and anti-proliferative substances do not annul themselves. Rather, different cell types or different biological entities react differently to different substances.

The invention is based on investigations of the inventors which suggest that “proliferative” nanoparticles of certain substances or combinations of substances are obviously in the position to selectively support the proliferation of specific cell types, while they hardly or not at all support the expansion of different cells (e.g. connective tissue) or germs. Hence, the presence of these substances does not lead to an increased risk of inflammation. It is also surprising that the combined presence of proliferative and anti-proliferative substances may, for example, significantly improve the acceptance and long-term stability of an implant. Obviously, suitable anti-proliferative substances may prevent the undesired attachment of certain cells, while the proliferative substances support the desired enclosing of the implant with different cell types. Thus, by suitably selecting a proliferatively active and an anti-proliferatively active substance, the proliferation of one cell type may selectively be supported, and the proliferation of a different cell type may be attenuated or prevented. However, the biologically active device of the present invention may not only be used as an implant or for an implant. Rather, it is also possible to use it for cell differentiation in a mixed culture of cells, for example for stem cell differentiation. Should this be done in-vitro, the device could be part of a petri dish in which the mixed culture is received. By the selection of one or several suitable nanoparticles which are proliferatively active on certain biological materials (for example cell lines or cell types), it is possible to selectively favor the growth or attachment of this “responsive” material. In an ideal case, a pure culture of a certain biological material (for example, a certain stem cell line) could be obtained in this way. By means of the anti-proliferatively active nanoparticles of a second substance, the growth of different cell types could be suppressed, thereby further supporting cell selection.

Preferably, the nanoparticles of the at least one proliferatively active material are metal or metal ion releasing nanoparticles which have a proliferative effect on particular tissue by releasing ions.

For example, these proliferatively active nanoparticles could comprise titanium, iron, magnesium, and/or oxides of these metals. The nanoparticles could also consist of a pure metal. There are indications that certain substances, such as iron, titanium, or magnesium have a neurotrophic effect, i.e. they specifically support the growth of nerve cells. This insight is valuable, for example for implants intended to provide for an electrical or optical signal transmission to or from a nerve, for example cochlear implants, brain implants, or pacemakers. Such implants require a very good contact between the nerve and an electrical (or optical) conductor in the implant. Very often such implants even provide pores through which the nerve cells may reach the electrical conductor. However, it is perturbing if connective tissue cells grow into the pores faster than the nerve cells, thereby clogging the pores. With the device of the present invention, this problem can be prevented by the substance(s) released from the device promoting the growth of the nerve cells, thereby ensuring that the nerve cells grow faster than the remaining tissue to the device, and in particular to an electrical conductor present in this device.

Alternatively (or in addition) to the metal nanoparticles, the main body of the device may comprise anorganic, proliferative nanoparticles. It is also possible that the polymer main body comprises proliferatively active nanoparticles of an organic material, a biological material (e.g. peptides), or a medical substance, such that the device could be used for drug delivery.

Preferred values for the size of the nanoparticles are sizes from 20 to 300 nm, in particular 60 to 200 nm. Via the surface-to-volume rate of the nanoparticles, and via their concentration in the polymer, their reservoir capacity and the rate of releasing the substances into the ambience of the device can be controlled. In this regard, a mean size of 20 to 300 nm, preferably 60 to 200 nm, has proven to be particularly advantageous. If the nanoparticles are even smaller, their reservoir capacity may be too small.

Nanoparticles for delivering anti-proliferatively or anti-adherently effective substances may, for example, be provided in the form of nanoparticles comprising silver, zinc, cobalt, aluminium, copper, and/or oxides of these metals, for example Co₂O, CuO, ZnO, ZnCl₂, or CuCl₂. Anti-proliferatively active nanoparticles may also be provided from an organic or from an anorganic substance, for example from antibiotics or other medical substances.

In principle, any suitable polymer could be used as the material for the main body of the device. It is useful, however, if the polymer main body comprises silicone, as silicone has turned out to be a particularly good material for implants with respect to its biocompatibility and its ability for storing nanoparticles. Further, it is important that the polymer material offers a possibility for the substances released from the nanoparticles to reach the surface of the device, and to enter from there into the ambience of the device. In this respect, too, silicone is particularly suited.

In a variant of the invention, the polymer main body is at least partially provided with at least one coating. The coating may serve to control or reduce the delivery of the bioactive substance(s) from the main body. Further, by arranging the coating on certain areas only, the delivery of bioactive substances from the device may be locally controlled. It is possible that nanoparticles of one or several materials are embedded into the coating. In this way, the device may obtain a two-step effect: the delivery of a bioactive substance from the main body can occur at a different rate (usually slower) than the delivery of a substance from the coating, since a longer distance must be covered from the main body to the surface.

It is advantageous if the nanoparticles embedded into the coating are different in their composition from the nanoparticles embedded in the polymer main body, in order to be able to provoke different tissue reactions.

In a useful variant, the coating has a barrier effect (i.e. it locally attenuates or prevents the release of substances from the device, or it prevents the undesired ingress of material from the ambience into the device), it has a biologic function, or it is biomimetic. The latter may, for example, mean that the coating has a surface structure or a surface roughness which is preferred by certain cell types, thereby further supporting the proliferative effect of the substance delivered from the device.

The coating does not necessarily have to be a monolayer, but it might also comprise several layers, which could have different functions. For example, nanoparticles of different materials could be embedded into each layer.

Particular advantages can be achieved if the device is an implant (or a part of an implant), preferably with a signal transmission means for an electrical or optical signal transmission to or from surrounding tissue, for example a cochlear implant. As means for an electrical signal transmission, an electrical conductor could be provided, which—for the sake of biocompatibility—could be made from platinum-iridium or from a different noble metal. As already explained, the signal transmission between the conductor in the implant and the nerve cells of the surrounding tissue could be significantly enhanced by favoring the proliferation of nerve cells to the implant, such that the nerve cells may grow towards the conductor or into its proximity before the space between the nerve and the conductor is filled by a different biological material, thereby increasing the impedance during the signal transmission.

It is also particularly advantageous if the device is used as or for a cardiovascular implant, in particular a heart flap, a polymer stent, or vascular prosthesis. In connection with cardiovascular implants, there is the problem of the so-called intima hyperplasia, wherein the implant is covered by smooth muscle cells (SMC cells) within a short time. First investigations show that the device of the present invention may selectively favor the growth or proliferation of endothelial cells by providing a certain magnesium concentration in the ambience of the device, without supporting the proliferation of SMC cells. Hence, a cell selection or cell differentiation is performed in the ambience of the implant, which favors endothelial cells. The implant could also be a port catheter temporarily remaining in the body, a microstent for opthalmology, or a ureter stent (i.e. a stent for the urinary passage).

As a polymer material for cardiovascular implants, the PES-material obtainable under the trade name Darcon has turned out to be efficient. In the present invention, the polymer material does not have to be pure, but the polymer material could comprise further additives, for example carbon fibers or other fibers for improving the mechanical properties.

The device could also be a catheter, a port catheter which does not remain in the body as an implant, a tracheal tube, a tracheal cannula, or a portion of these products.

The present invention is also related to a method of manufacturing a biologically active device. At first, nanoparticles of several different materials are generated and dispersed in an injection-moldable fluid, before the fluid is shaped by injection molding and curing to a polymer main body of the device, such that the nanoparticles are dispersed or embedded in the bulk of the polymer main body. In particular, the nanoparticles could be distributed homogeneously in the main body. According to the invention, the nanoparticles are generated by arranging at least two substrates of different material in a vessel filled with a fluid material (for example, a monomer or a resolvent), and by generating the nanoparticles by abrasion from the surface of the substrates within the fluid by means of a laser (e.g. by means of pulsed laser radiation).

An advantage of the method of the present invention is that the device is producible in a rather easy manner, since the main body defines both the shape of the device and its biological effect. Further, the device is adaptable ideally to different use purposes by selecting the material and size of the nanoparticles, as well as by the selection of a polymer. It could also be shown that the shape and size of the nanoparticles generated from the substrate are precisely selectable by adjusting the laser parameters (pulse duration, wave length, fluence, etc).

An important advantage of the inventive method is that it does not require the separate generation and subsequent mixing of two or more colloids of different substances, which might lead to several problems, including an increased volume, an insufficient, inhomogeneous mixing, or a potential co-precipitation (i.e. a flocculation of one or both nanoparticle types). According to the invention, the nanoparticles of different materials are rather generated in a single vessel, possibly even simultaneously or intermittently. The interaction of the laser radiation with the two or more native (i.e. freshly generated) nanoparticle types leads to unexpected advantages in comparison to the subsequent mixing. For example, it has turned out that one type of particles (for example, plasmonresonant particles) may transfer absorbed energy onto the other type, such that the second type of particles becomes smaller and/or more stable compared to separately generated particles. It has also turned out that the nanoparticles have a higher reactivity when freshly generated in comparison to later times, such that they may be connected with the particles freshly generated in the same vessel in a controlled manner (for example, by sorbing or by alloying). The colloidal stability is maintained (i.e. there is a reduced tendency for flocculation, agglomeration, or sedimentation). Further, the problems which otherwise occur during mixing of different colloids are avoided.

As described above, the nanoparticles of at least one material may preferably be proliferatively active when embedding the device into a tissue.

It is also possible that the device comprises nanoparticles of at least one material which is anti-proliferatively or anti-adherently active after embedding the material into a tissue, possibly also in combination with nanoparticles of a different, proliferatively active material.

The generation of nanoparticles may preferably be done by abrasion from the surface of a substrate by means of a short pulse or ultra short pulse laser, i.e. with pulse durations in the range of nanoseconds (ns), picoseconds (ps), or femtoseconds (fs). With such ultra-short laser pulses, the nanoparticles can be obtained from the substrate in a stoichiometric manner, since the short duration of the pulses avoids a thermal effect on the substrate. Further, a thermal influence on the fluid surrounding the substrate is avoided.

In the method of the present invention, the abrasion may be performed in an alternate manner from the two or more substrates. If more than two substrates are present, the laser may be guided in a random manner or repeatedly in a predetermined pattern across the substrates. In this way, the nanoparticles of different materials are generated substantially simultaneously, which leads to an excellent mixing.

In particular, each laser pulse may be guided onto a different substrate than the preceding laser pulse.

It is beneficial if the laser pulse or the laser radiation, respectively, can be directed onto the different substrates by means of a controllable deflection means with one or two pivotable mirrors, for example a galvanometric scanner.

The fluid material in which the nanoparticles are generated could itself be an injection-moldable fluid (i.e. a monomer solution), or it could be replaced by an injection-moldable fluid after generation of the nanoparticles.

The inventive method is characterized by comprising an injection-molding step. The injection molding offers the advantage of being able to simultaneously produce a plurality of similar or differently shaped polymer main bodies. This method of production reduces the costs of producing the devices considerably.

The polymer main body may be provided on at least a part of its surface with a coating, in order to control the release of substances from the main body, or in order to allow the release of further substances embedded in the coating.

In the following, a preferred embodiment of the invention will be described with reference to a drawing.

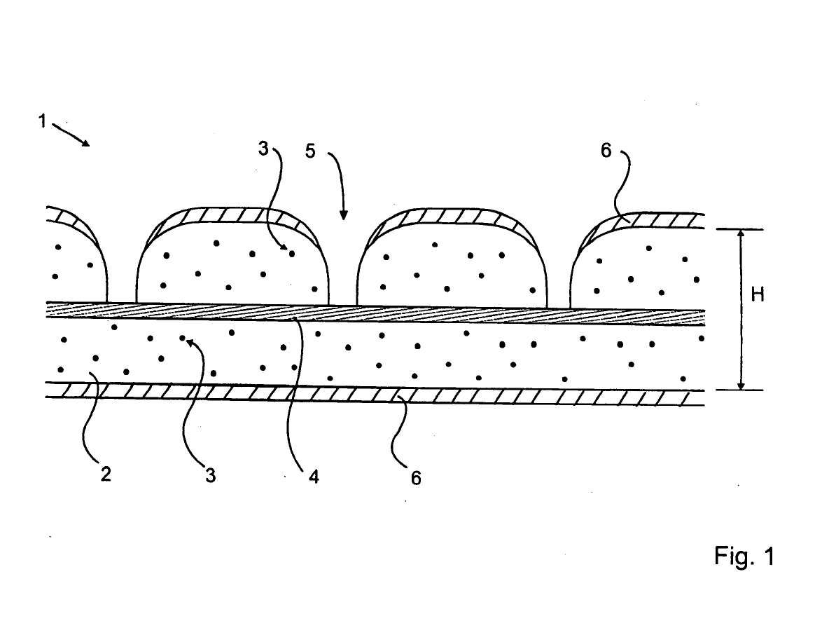

FIG. 1 shows a section through an embodiment of a device according to the invention,

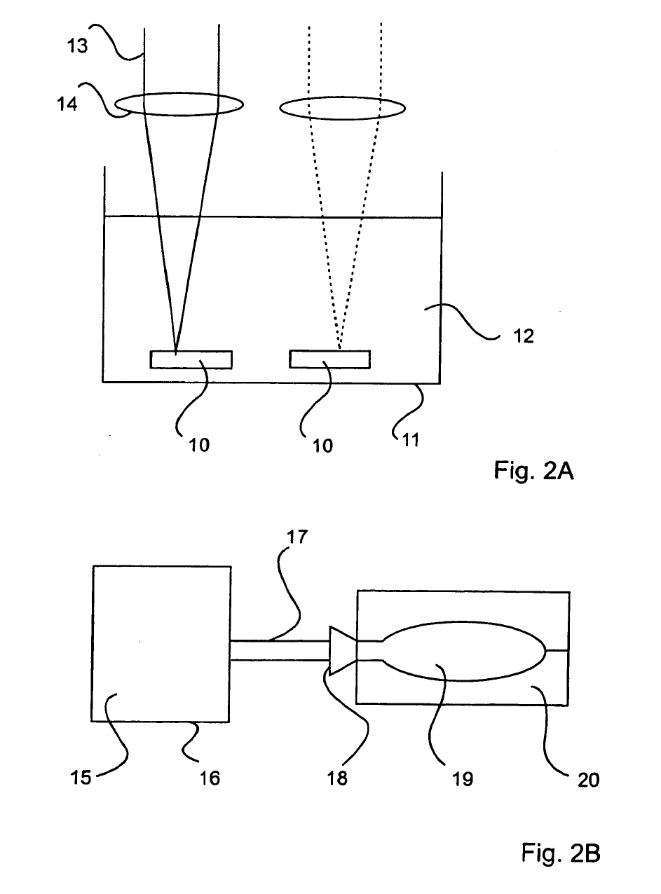

FIG. 2A is a schematic representation of the generation of nanoparticles,

FIG. 2B is a schematic representation of injection-molding the device of the present invention.

### DETAILED DESCRIPTION OF THE PREFERRED EMBODIMENT

FIG. 1 shows a section through an embodiment of a biologically active device 1 of the present invention. The device 1 comprises a polymer main body 2, which is preferably injection-molded from silicone or Darcon. The main body 2 may for example be plate-shaped or cylindrical. Depending on the application, it may have a height H from about 1 mm to several centimeters.

“Bioactive” nanoparticles 3 of different materials with a size of 60 to 200 nm are homogeneously dispersed in the bulk of the main body 2. Across their surface, the nanoparticles 3 release a substance which diffuses out of the device 1 and which acts on biological material (not shown) in the ambience of the device 1 in a “bioactive” manner. While the nanoparticles 3 of one material release a substance which acts in a proliferative way on the biological material, the nanoparticles 3 of a different material release a substance which acts in an anti-proliferative or anti-adherent manner. For this purpose, the nanoparticles 3 of one type may consist of calcium, calcium salt, calcium phosphate, hydroxylapatite, magnesium, magnesium salt, titanium or titanium oxide, and release calcium, magnesium, or titanium ions, respectively, which may have a positive effect on nerve cells, for example in a neurotropic manner. As anti-proliferative or anti-adherent nanoparticles, nanoparticles 3 of silver, copper, or zinc(oxide) may be present, which are also dispersed in the main body 2.

A conductor 4 is embedded, for example inserted, into the bulk of the main body 2. The conductor 4 serves to deliver electrical (or optical) signals into the biological ambience of the device 1, or to receive electrical (or optical) signals from there. If the device 1 is used as a cochlear implant, for example, signals may be transmitted to the acoustic nerves.

The main body 2 comprises pores 5 in which the surface of the main body 2 is retreated to such an extent that the electrical conductor 4 is exposed. In this way, the conductor 4 may be directly contacted by nerve cells. For this purpose, however, it is a precondition that the nerve cells grow into the pores 5. This is achieved by at least one substance, released from the nanoparticles 3, which selectively acts on the nerve cells in a proliferative manner such that growth of these cells into the pores 5 is favored. The substances released from the “anti-proliferative” nanoparticles may, for example, act on cells other than nerve cells in an anti-proliferative manner, thereby preventing that other cells (for example connective tissue) occupy the pores 5 before the nerve cells.

The surface of the device 1 is provided with a coating 6, but not in the area of the pores 5. The coating 6 influences the egression of proliferative substances from the main body 2. Hence, the concentration of these substances is particularly high in the area of the pores 5, such that the nerve cells preferably grow into the direction of the pores, and into the pores themselves. By suitably arranging the coating 6, an anisotropic distribution of the bioactive substance(s) outside the device 1 may be adjusted. The coating 6 itself may comprise bioactive nanoparticles 3, which may act in a proliferative and/or anti-proliferative manner on particular tissue or cell types.

FIG. 2 generally shows a preferred embodiment of the manufacturing method of the present invention.

As shown in FIG. 2A, one or several (here: two) substrates 10 are accommodated in a vessel 11 which is filled by a fluid material 12. Each substrate 10 is a material from which nanoparticles 3 are subsequently obtained. The fluid material 12 may already be an injection-moldable fluid, or a solvent which is replaced by an injection-moldable fluid in one or several subsequent steps.

A beam 13 of an ultra-short pulse laser is focused via a focusing optics 14 on, or in the vicinity of, the surface of the substrate 10. The laser pulses release nanoparticles 3 from the substrate 10, which nanoparticles are instantaneously dispersed and stabilized in the fluid material 12. If another substrate 10 of a different material is present, nanoparticles may be obtained from this other substrate 10 by a suitable deflection of the laser pulses. Via a deflecting means (not shown), for example, the laser beam may be intermittently guided onto the two or more substrates, such that the laser pulses impinge on the two substrates in an alternate manner.

The fluid material 12 in which the nanoparticles 3 are dispersed may be replaced, if necessary, by an injection-moldable prepolymer fluid 15 (such that the nanoparticles are now dispersed in the injection-moldable fluid) and subsequently accommodated in a reservoir 16. FIG. 2B shows that the injection-moldable fluid 15 is guided from the reservoir 16 via a conduit 17 to an injection nozzle 18 in order to be guided through the nozzle 18 into the cavity 19 of a multi-part injection molding tool 20.

The injected fluid 15 cures in the cavity 19 to a polymer main body 2, in the bulk of which the nanoparticles 3 are dispersed or embedded, respectively. If desired, the main body 2 may be provided after curing with a coating 6. The resulting device 1 of the invention may then, for example, be used as an implant, or in a cell culture for differentiating between different cell types.

Starting from the embodiment discussed in detail, the device 1 and the method of the present invention may be amended in several ways, in particular with respect to the materials used. Further, the molding tool might comprise a plurality of mold cavities in which a corresponding number of devices of the present invention can be simultaneously produced.

It is intended to cover in the appended claims all such modifications and variations as fall within the true spirit and scope of the invention.

1. A biologically active device with a main body made from a polymer in which bioactive nanoparticles of one or several materials are embedded, wherein the nanoparticles of at least one material are configured to release a substance which may act in a proliferative manner on biological material in the ambience of the device, wherein the device, further to the nanoparticles releasing a proliferatively active substance, also comprises nanoparticles of at least one further material which are configured to release a substance which may act in an anti-proliferative or anti-adherent manner on biological material in the ambience of the device.

2. The device according to claim 1, wherein the nanoparticles of the at least one proliferatively active material are metallic nanoparticles, or metal ion releasing nanoparticles.

3. The device according to claim 2, wherein the proliferatively active nanoparticles comprise titanium, iron, magnesium, and/or oxides of these metals.

4. The device according to claim 1, wherein the nanoparticles of the at least one proliferatively active material are anorganic nanoparticles.

5. The device according to claim 1, wherein the polymer main body comprises nanoparticles of an organic material, a biological material, or a medical substance.

6. The device according to claim 1, wherein the nanoparticles of one or several materials have a size in the range of 20 to 300 nm, preferably from 60 to 200 nm.

7. The device according to claim 1, wherein the anti-proliferatively active nanoparticles comprise silver, zinc, cobalt, aluminium, copper or oxides of these metals.

8. The device according to claim 1, wherein anti-proliferatively active nanoparticles of an organic or an anorganic material are present in the polymer main body.

9. The device according to claim 1, wherein the polymer main body comprises silicone.

10. The device according to claim 1, wherein at least a portion of the polymer main body is provided with at least one coating.

11. The device according to claim 10, wherein nanoparticles of one or several materials are embedded into the coating.

12. The device according to claim 11, wherein the nanoparticles embedded in the coating are different in their composition from the nanoparticles embedded in the polymer main body.

13. The device according to claim 10, wherein the coating has a barrier function, a biologic function, or a biomimetic function.

14. The device according to claim 10, wherein the coating comprises several layers in which nanoparticles of different materials are embedded, respectively.

15. The device according to claim 1, wherein the device is an implant or a part of an implant.

16. The device according to claim 15, wherein the implant is selected from a group comprising: a cochlear implant, a cardiovascular implant, a heart flap, a port catheter, a polymer stent, a vascular prosthesis, a microstent for opthalmology, and a ureter stent.

17. The device according to claim 15, wherein the device is an implant comprising a signal transmission means for an electrical or optical transmission to or from the surrounding tissue.

18. The device according to claim 1, wherein the device is a catheter, a port catheter, a tracheal tube, a tracheal cannula, or a part of these products.

19. A method for manufacturing a biologically active device with a main body made from a polymer, wherein nanoparticles of several different materials are dispersed in an injection-moldable fluid, and the fluid is shaped by injection molding and curing to the polymer main body, such that the nanoparticles are dispersed in the volume of the polymer main body,
wherein the nanoparticles are generated by arranging at least two substrates of different materials in a vessel filled with a fluid material, and by generating the nanoparticles by abrasion from the surface of the substrates in the fluid by means of laser radiation.

20. The method according to claim 19, wherein the nanoparticles of at least one material are proliferatively active when the device is embedded into a tissue.

21. The method according to claim 19, wherein the nanoparticles of at least one material are anti-proliferatively or anti-adherently active when the device is embedded into a tissue.

22. The device according to claim 19, wherein the abrasion is performed in an alternate manner from two or more substrates.

23. The method according to claim 19, wherein the nanoparticles are generated by abrasion from the surface of the substrates by means of pulsed laser radiation from a short pulse laser, or from an ultra-short pulse laser.

24. The method according to claim 23, wherein each laser pulse is directed onto a different substrate than the preceding laser pulse, respectively.

25. The method according to claim 19, wherein the laser radiation is directed onto the different substrates by means of a controllable deflection means with one or two pivotable mirrors.

26. The method according to claim 19, wherein the fluid material, in which the nanoparticles are generated, is itself an injection-moldable fluid, or is replaced by an injection-moldable fluid.

27. The method according to claim 19, wherein a plurality of polymer main bodies is simultaneously produced in the injection molding step.

28. The method according to claim 19, wherein the polymer main body is provided with a coating.

29. Use of a device according to claim 1 for in-vivo or in-vitro cell differentiation.
